(19) United States
(12) Patent Application Publication
(10) Pub. No.: US 2004/0023230 A1
(43) Pub. Date: Feb. 5, 2004

(54) Method and nucleic acids for pharmacogenomic methylation analysis

(76) Inventors: Alexander Olek, Berlin (DE); Christian Piepenbrock, Berlin (DE); Kurt Berlin, Stahnsdorf (DE)

Correspondence Address: DAVIDSON, DAVIDSON & KAPPEL, LLC, 485 SEVENTH AVENUE, 14TH FLOOR, NEW YORK, NY, 10018, US

(21) Appl. No.: 10/257,166

(22) PCT Filed: Jun. 29, 2001
(86) PCT No.: PCT/EP01/07470

(51) Int. Cl. C12Q 1/68; C07H 21/04

(52) U.S. Cl. 435/6; 435/69.1; 435/320.1; 435/325; 536/23.2

(57) ABSTRACT

The present invention relates to the chemically modified genomic sequences of genes associated with pharmacogenomics, to oligonucleotides and/or PNA-oligomers for detecting the cytosine methylation state of genes associated with pharmacogenomics which are directed against the sequence, as well as to a method for ascertaining genetic and/or epigenetic parameters of genes associated with pharmacogenomics.

1. Field of the Invention

The levels of observation that have been well studied by the methodological developments of recent years in molecular biology, are the genes themselves, the translation of these genes into RNA, and the resulting proteins. The question of which gene is switched on at which point in the course of the development of an individual, and how the activation and inhibition of specific genes in specific cells and tissues are controlled is correlatable to the degree and character of the methylation of the genes or of the genome. In this respect, pathogenic conditions may manifest themselves in a changed methylation pattern of individual genes or of the genome.

The present invention relates to nucleic acids, oligonucleotides, PNA-oligomers and to a method for the analysis of genetic and/or epigenetic parameters of genes associated with pharmacogenomics and, in particular, with the methylation status thereof.

2. Prior Art

5-methylcytosine is the most frequent covalent base modification in the DNA of eukaryotic cells. It plays a role, for example, in the regulation of the transcription, in genetic imprinting, and in tumorigenesis.

Therefore, the identification of 5-methylcytosine as a component of genetic information is of considerable interest. However, 5-methylcytosine positions cannot be identified by sequencing since 5-methylcytosine has the same base pairing behavior as cytosine. Moreover, the epigenetic information carried by 5-methylcytosine is completely lost during PCR amplification.

A relatively new and currently the most frequently used method for analyzing DNA for 5-methylcytosine is based upon the specific reaction of bisulfite with cytosine which, upon subsequent alkaline hydrolysis, is converted to uracil which corresponds to thymidine in its base pairing behavior. However, 5-methylcytosine remains unmodified under these conditions. Consequently, the original DNA is converted in such a manner that methylcytosine, which originally could not be distinguished from cytosine by its hybridization behavior, can now be detected as the only remaining cytosine using “normal” molecular biological techniques, for example, by amplification and hybridization or sequencing. All of these techniques are based on base pairing which can now be fully exploited. In terms of sensitivity, the prior art is defined by a method which encloses the DNA to be analyzed in an agarose matrix, thus preventing the diffusion and renaturation of the DNA (bisulfite only reacts with single-stranded DNA), and which replaces all precipitation and purification steps with fast dialysis (Olek A, Oswald J, Walter J. A modified and improved method for bisulphite based cytosine methylation analysis. Nucleic Acids Res. Dec. 15, 1996;24(24):5064-6). Using this method, it is possible to analyze individual cells, which illustrates the potential of the method. However, currently only individual regions of a length of up to approximately 3000 base pairs are analyzed, a global analysis of cells for thousands of possible methylation events is not possible. However, this method cannot reliably analyze very small fragments from small sample quantities either. These are lost through the matrix in spite of the diffusion protection.

An overview of the further known methods of detecting 5-methylcytosine may be gathered from the following review article: Rein, T., DePamphilis, M. L., Zorbas, H., Nucleic Acids Res. 1998, 26, 2255.

To date, barring few exceptions (e.g., Zeschnigk M, Lich C, Buiting K, Doerfler W, Horsthemke B. A single-tube PCR test for the diagnosis of Angelman and Prader-Willi syndrome based on allelic methylation differences at the SNRPN locus. Eur J Hum Genet. 1997 March-April;5(2):94-8) the bisulfite technique is only used in research. Always, however, short, specific fragments of a known gene are amplified subsequent to a bisulfite treatment and either completely sequenced (Olek A, Walter J. The pre-implantation ontogeny of the H19 methylation imprint. Nat Genet. 1997 November;17(3):275-6) or individual cytosine positions are detected by a primer extension reaction (Gonzalgo M L, Jones Pa. Rapid quantitation of methylation differences at specific sites using methylation-sensitive single nucleotide primer extension (Ms-SNuPE). Nucleic Acids Res. Jun. 15, 1997;25(12):2529-31, WO 95/00669) or by enzymatic digestion (Xiong Z, Laird P W. COBRA: a sensitive and quantitative DNA methylation assay. Nucleic Acids Res. Jun. 15, 1997;25(12):2532-4). In addition, detection by hybridization has also been described (Olek et al., WO 99/28498).

Further publications dealing with the use of the bisulfite technique for methylation detection in individual genes are: Grigg G, Clark S. Sequencing 5-methylcytosine residues in genomic DNA. Bioessays. 1994 June;16(6):431-6, 431; Zeschnigk M, Schmitz B, Dittrich B, Buiting K, Horsthemke B, Doerfler W. Imprinted segments in the human genome: different DNA methylation patterns in the Prader-Willi/Angelman syndrome region as determined by the genomic sequencing method. Hum Mol Genet. 1997 March;6(3):387-95; Feil R, Charlton J, Bird A P, Walter J, Reik W. Methylation analysis on individual chromosomes: improved protocol for bisulphite genomic sequencing. Nucleic Acids Res. Feb. 25, 1994;22(4):695-6; Martin V, Ribieras S, Song-Wang X, R10 M C, Dante R. Genomic sequencing indicates a correlation between DNA hypomethylation in the 5′ region of the pS2 gene and its expression in human breast cancer cell lines. Gene. May 19, 1995;157(1-2):261-4; WO 97/46705, WO 95/15373 and WO 97/45560.

An overview of the Prior art in oligomer array manufacturing can be gathered from a special edition of Nature Genetics (Nature Genetics Supplement, Volume 21, January 1999), published in January 1999, and from the literature cited therein.

Fluorescently labeled probes are often used for the scanning of immobilized DNA arrays. The simple attachment of Cy3 and Cy5 dyes to the 5′-OH of the specific probe are particularly suitable for fluorescence labels. The detection of the fluorescence of the hybridized probes may be carried out, for example via a confocal microscope. Cy3 and Cy5 dyes, besides many others, are commercially available.

Matrix Assisted Laser Desorption Ionization Mass Spectrometry (MALDI-TOF) is a very efficient development for the analysis of biomolecules (Karas M, Hillenkamp F. Laser desorption ionization of proteins with molecular masses exceeding 10,000 daltons. Anal Chem. Oct. 15, 1988;60(20):2299-301). An analyte is embedded in a light-absorbing matrix. The matrix is evaporated by a short laser pulse thus transporting the analyte molecule into the vapor phase in an unfragmented manner. The analyte is ionized by collisions with matrix molecules. An applied voltage accelerates the ions into a field-free flight tube. Due to their different masses, the ions are accelerated at different rates. Smaller ions reach the detector sooner than bigger ones.

MALDI-TOF spectrometry is excellently suited to the analysis of peptides and proteins. The analysis of nucleic acids is somewhat more difficult (Gut I G, Beck S. DNA and Matrix Assisted Laser Desorption Ionization Mass Spectrometry. Current Innovations and Future Trends. 1995, 1; 147-57). The sensitivity to nucleic acids is approximately 100 times worse than to peptides and decreases disproportionally with increasing fragment size. For nucleic acids having a multiply negatively charged backbone, the ionization process via the matrix is considerably less efficient. In MALDI-TOF spectrometry, the selection of the matrix plays an eminently important role. For the desorption of peptides, several very efficient matrixes have been found which produce a very fine crystallization. There are now several responsive matrixes for DNA, however, the difference in sensitivity has not been reduced. The difference in sensitivity can be reduced by chemically modifying the DNA in such a manner that it becomes more similar to a peptide. Phosphorothioate nucleic acids in which the usual phosphates of the backbone are substituted with thiophosphates can be converted into a charge-neutral DNA using simple alkylation chemistry (Gut IG, Beck S. A procedure for selective DNA alkylation and detection by mass spectrometry. Nucleic Acids Res. Apr. 25, 1995;23(8):1367-73). The coupling of a charge tag to this modified DNA results in an increase in sensitivity to the same level as that found for peptides. A further advantage of charge tagging is the increased stability of the analysis against impurities which make the detection of unmodified substrates considerably more difficult.

Pharmacogenomics is the science of utilising human genetic variation to optimise patient treatment and drug design and discovery. An individual's genetic make up affects each stage of drug response: absorption, metabolism, transport to the target molecule, structure of the intended and/or unintended target molecules, degradation and excretion.

Pharmacogenomics provides the basis for a new generation of personalized pharmaceuticals, the targeting of drug therapies to genetic subpopulations. Currently drugs are developed to benefit the widest possible populations. However the variations in drug reactions attributed to genetic variation are increasingly been taken into account when developing new drugs. There are multiple benefits to such an approach to drug design. The development of genetic tests may reduce the need for the standard trial and error method of drug prescription. Targeted prescriptions would further reduce the incidence of adverse drug reactions, which are estimated to be the fifth ranking cause of death in the United States. Furthermore, dosage decisions can be made on a more informed basis than currently used parameters such as age, sex and weight. Drug discovery and approval processes will likely be speeded up by the specific genetic targeting of candidate drugs. Moreover, this may allow the revival of previously failed candidate drugs. Overall it is expected that the development of personalized pharmaceuticals will reduce the costs of healthcare.

Several candidate genes have been identified that influence drug reactions, most notably the cytochrome P450 family. The cytochrome P450 monooxygenase system is responsible for a large proportion of drug metabolism in the body, furthermore it is also responsible for the activation of procarcinogens and promutagens. In particular, the CYP2D6, 3A4/3A5, 1A2, 2E1, 2C9, and 2C19 genes have been identified as key regulators of drug response. For example, homozygozity for the CYP2D6 null allele has a frequency of 1% to 2% in Asians, 5% in African Americans, and 6% to 10% in Caucasian populations. This genotype exhibits reduced degradation and excretion of many drugs including debrisoquine, metaprolol, nortrptyline and propafone. Another important member of the family is the CYP2C9 gene. It metabolizes a variety of important drugs, including ibuprofen, naproxen, piroxicam, tetrahydrocannabinol, phenytoin, tolbutamide, and S-warfarin. Substitutions in codons 144 and 359 result in a 5-fold decline in metabolic activity. Although the frequency of such mutations is unknown it has been estimated at 25% heterozygosity in the caucasian population.

A particular target in pharmacogenomics is the characterisation of single nucleotide polymorphisms and their effects on drug response. For example, response to the drugs pravastatin (treatment of high cholesterol), Clozapine (schizophrenia treatment) and procainamide (heart arrythymia) have all been shown to be affected by SNPs.

The benefits of pharmacogenetically developed drugs are of particular interest in diseases such as cancer, where efficacy and side effects show wide variation. Furthermore, the genetic basis of diseases such as cancer makes them appropriate targets. The first commercially available drug targeted at a specific genotype was Herceptin, a humanized monoclonal antibody for the treatment of metastatic breast cancer. Herceptin is useful in the 25%-30% of breast cancer patients who over express the HER2 (human epidermal growth factor receptor 2) protein. Alternatively, pharmacogenomics is also used to screen patients who may have adverse reactions to drugs. For example, azathioprine and mercaptopurine are commonly used treatments for acute lymphoblastic leukaemia in children. However, patients deficient in thiopurine methyl transferase are unable to adequately metabolize mercaptopurine and are at risk of developing life threatening myelosuppression.

Genomic DNA is obtained from DNA of cell, tissue or other test samples using standard methods. This standard methodology is found in references such as Fritsch and Maniatis eds., Molecular Cloning: A Laboratory Manual, 1989.

### DESCRIPTION

The object of the present invention is to provide the chemically modified DNA of genes associated with pharmacogenomics, as well as oligonucleotides and/or PNA-oligomers for detecting cytosine methylations, as well as a method which is particularly suitable for the analysis of genetic and epigenetic parameters of genes associated with pharmacogenomics. The present invention is based on the discovery that genetic and epigenetic parameters and, in particular, the cytosine methylation pattern of genes associated with pharmacogenomics are particularly suitable for the development and analysis of novel drugs and therapies.

This objective is achieved according to the present invention using a nucleic acid containing a sequence of at least 18 bases in length of the chemically pretreated DNA of genes associated with pharmacogenomics according to one of Seq. ID No.1 through Seq. ID No.174 and sequences complementary thereto and/or of a segment of the chemically pretreated DNA of genes associated with pharmacogenomics according to one of the sequences according to table 1. In the table, after the listed gene designations, the respective data bank numbers (accession numbers) are specified which define the appertaining gene sequences as unique. GenBank was used as the underlying data bank, which is located at internet address http://www.ncbi.nlm.nih.gov

The chemically modified nucleic acid could heretofore not be connected with the ascertainment of genetic and epigenetic parameters.

The object of the present invention is further achieved by an oligonucleotide or oligomer for detecting the cytosine methylation state in chemically pretreated DNA, containing at least one base sequence having a length of at least 13 nucleotides which hybridizes to a chemically pretreated DNA of genes associated with pharmacogenomics according to Seq. ID No.1 through Seq. ID No.174 and sequences complementary thereto and/or of a segment of the chemically pretreated DNA of genes associated with pharmacogenomics according to one of the sequences according to table 1. The oligomer probes according to the present invention constitute important and effective tools which, for the first time, make it possible to ascertain the genetic and epigenetic parameters of genes associated with pharmacogenomics. The base sequence of the oligomers preferably contains at least one CpG dinucleotide. The probes may also exist in the form of a PNA (peptide nucleic acid) which has particularly preferred pairing properties. Particularly preferred are oligonucleotides according to the present invention in which the cytosine of the CpG dinucleotide is the 5^{th}-9^{th} nucleotide from the 5′-end of the 13-mer; in the case of PNA-oligomers, it is preferred for the cytosine of the CpG dinucleotide to be the 4^{th}-6^{th} nucleotide from the 5′-end of the 9-mer.

The oligomers according to the present invention are normally used in so called “sets” which contain at least one oligomer for each of the CpG dinucleotides of the sequences of Seq. ID No.1 through Seq. ID No.174 and sequences complementary thereto and/or of a segment of the chemically pretreated DNA of genes associated with pharmacogenomics according to one of the sequences according to table 1. Preferred is a set which contains at least one oligomer for each of the CpG dinucleotides from one of Seq. ID No. 1 through Seq. ID No.174 and sequences complementary thereto and/or of a segment of the chemically pretreated DNA of genes associated with pharmacogenomics according to one of the sequences according to table 1.

Moreover, the present invention makes available a set of at least two oligonucleotides which can be used as so-called “primer oligonucleotides” for amplifying DNA sequences of one of Seq. ID No.1 through Seq. ID No.174 and sequences complementary thereto and/or of a segment of the chemically pretreated DNA of genes associated with pharmacogenomics according to one of the sequences according to table 1, or segments thereof.

In the case of the sets of oligonucleotides according to the present invention, it is preferred that at least one oligonucleotide is bound to a solid phase. Furthermore, it is preferred that all the oligonucleotides of a set are bound to a solid phase.

The present invention moreover relates to a set of at least 10 n (oligonucleotides and/or PNA-oligomers) used for detecting the cytosine methylation state in chemically pretreated genomic DNA (Seq. ID No.1 through Seq. ID No.174 and sequences complementary thereto and/or of a segment of the chemically pretreated DNA of genes associated with pharmacogenomics according to one of the sequences according to table 1). These probes enable the determination of genetic and epigenetic parameters of genes associated with pharmacogenomics. The set of oligomers may also be used for detecting single nucleotide polymorphisms (SNPs) in the chemically pretreated DNA of genes associated with pharmacogenomics according to one of Seq. ID No.1 through Seq. ID No.174 and sequences complementary thereto and/or of a segment of the chemically pretreated DNA of genes associated with pharmacogenomics according to one of the sequences according to table 1.

According to the present invention, it is preferred that an arrangement of different oligonucleotides and/or PNA-oligomers (a so-called “array”) made available by the present invention is present in a manner that it is likewise bound to a solid phase. This array of different oligonucleotide- and/or PNA-oligomer sequences can be characterized in that it is arranged on the solid phase in the form of a rectangular or hexagonal lattice. The solid phase surface is preferably composed of silicon, glass, polystyrene, aluminium, steel, iron, copper, nickel, silver, or gold. However, nitrocellulose as well as plastics such as nylon which can exist in the form of pellets or also as resin matrices are possible as well.

Therefore, a further subject matter of the present invention is a method for manufacturing an array fixed to a carrier material for analysis in connection with diseases associated with pharmacogenomics in which method at least one oligomer according to the present invention is coupled to a solid phase. Methods for manufacturing such arrays are known, for example, from U.S. Pat. No. 5,744,305 by means of solid-phase chemistry and photolabile protecting groups.

A further subject matter of the present invention relates to a DNA chip for the analysis of genetic and epigenetic parameters of genes associated with pharmacogenomics which contains at least one nucleic acid according to the present invention. DNA chips are known, for example, for U.S. Pat. No. 5,837,832.

Moreover, a subject matter of the present invention is a kit which may be composed, for example, of a bisulfite-containing reagent, a set of primer oligonucleotides containing at least two oligonucleotides whose sequences in each case correspond or are complementary to an 18 base long segment of the base sequences specified in the appendix (Seq. ID No. 1 through Seq. ID No.174 and sequences complementary thereto and/or of a segment of the chemically pretreated DNA of genes associated with pharmacogenomics according to one of the sequences according to table 1), oligonucleotides and/or PNA-oligomers as well as instructions for carrying out and evaluating the described method. However, a kit along the lines of the present invention can also contain only part of the aforementioned components.

The present invention also makes available a method for ascertaining genetic and/or epigenetic parameters of genes associated with pharmacogenomics by analyzing cytosine methylations and single nucleotide polymorphisms, including the following steps:

In the first step of the method, a genomic DNA sample is chemically treated in such a manner that cytosine bases which are unmethylated at the 5′-position are converted to uracil, thymine, or another base which is dissimilar to cytosine in terms of hybridization behavior. This will be understood as ‘chemical pretreatment’ hereinafter.

The genomic DNA to be analyzed is preferably obtained form usual sources of DNA such as cells or cell components, for example, cell lines, biopsies, blood, sputum, stool, urine, cerebral-spinal fluid, tissue embedded in paraffin such as tissue from eyes, intestine, kidney, brain, heart, prostate, lung, breast or liver, histologic object slides, or combinations thereof.

The above described treatment of genomic DNA is preferably carried out with bisulfite (hydrogen sulfite, disulfite) and subsequent alkaline hydrolysis which results in a conversion of non-methylated cytosine nucleobases to uracil or to another base which is dissimilar to cytosine in terms of base pairing behavior.

Fragments of the chemically pretreated DNA are amplified, using sets of primer oligonucleotides according to the present invention, and a, preferably heat-stable polymerase. Because of statistical and practical considerations, preferably more than ten different fragments having a length of 100-2000 base pairs are amplified. The amplification of several DNA segments can be carried out simultaneously in one and the same reaction vessel. Usually, the amplification is carried out by means of a polymerase chain reaction (PCR).

In a preferred embodiment of the method, the set of primer oligonucleotides includes at least two olignonucleotides whose sequences are each reverse complementary or identical to an at least 18 base-pair long segment of the base sequences specified in the appendix (Seq. ID No 0.1 through Seq. ID No.174 and sequences complementary thereto and/or of a segment of the chemically pretreated DNA of genes associated with pharmacogenomics according to one of the sequences according to table 1). The primer oligonucleotides are preferably characterized in that they do not contain any CpG dinucleotides.

According to the present invention, it is preferred that at least one primer oligonucleotide is bonded to a solid phase during amplification. The different oligonucleotide and/or PNA-oligomer sequences can be arranged on a plane solid phase in the form of a rectangular or hexagonal lattice, the solid phase surface preferably being composed of silicon, glass, polystyrene, aluminium, steel, iron, copper, nickel, silver, or gold, it being possible for other materials such as nitrocellulose or plastics to be used as well.

The fragments obtained by means of the amplification can carry a directly or indirectly detectable label. Preferred are labels in the form of fluorescence labels, radionuclides, or detachable molecule fragments having a typical mass which can be detected in a mass spectrometer, it being preferred that the fragments that are produced have a single positive or negative net charge for better detectability in the mass spectrometer. The detection may be carried out and visualized by means of matrix assisted laser desorption/ionization mass spectrometry (MALDI) or using electron spray mass spectrometry (ESI).

The amplificates obtained in the second step of the method are subsequently hybridized to an array or a set of oligonucleotides and/or PNA probes. In this context, the hybridization takes place in the manner described in the following. The set of probes used during the hybridization is preferably composed of at least 10 oligonucleotides or PNA-oligomers. In the process, the amplificates serve as probes which hybridize to oligonucleotides previously bonded to a solid phase. The non-hybridized fragments are subsequently removed. Said oligonucleotides contain at least one base sequence having a length of 13 nucleotides which is reverse complementary or identical to a segment of the base sequences specified in the appendix, the segment containing at least one CpG dinucleotide. The cytosine of the CpG dinucleotide is the 5^{th} to 9^{th} nucleotide from the 5′-end of the 13-mer. One oligonucleotide exists for each CpG dinucleotide. Said PNA-oligomers contain at least one base sequence having a length of 9 nucleotides which is reverse complementary or identical to a segment of the base sequences specified in the appendix, the segment containing at least one CpG dinucleotide. The cytosine of the CpG dinucleotide is the 4^{th} to 6^{th} nucleotide seen from the 5′-end of the 9-mer. One oligonucleotide exists for each CpG dinucleotide.

In the fourth step of the method, the non-hybridized amplificates are removed.

In the final step of the method, the hybridized amplificates are detected. In this context, it is preferred that labels attached to the amplificates are identifiable at each position of the solid phase at which an oligonucleotide sequence is located.

According to the present invention, it is preferred that the labels of the amplificates are fluorescence labels, radionuclides, or detachable molecule fragments having a typical mass which can be detected in a mass spectrometer. The mass spectrometer is preferred for the detection of the amplificates, fragments of the amplificates or of probes which are complementary to the amplificates, it being possible for the detection to be carried out and visualized by means of matrix assisted laser desorption/ionization mass spectrometry (MALDI) or using electron spray mass spectrometry (ESI).

The produced fragments may have a single positive or negative net charge for better detectability in the mass spectrometer. The aforementioned method is preferably used for ascertaining genetic and/or epigenetic parameters of genes associated with pharmacogenomics.

The oligomers according to the present invention or arrays thereof as well as a kit according to the present invention are intended to be used for the determination of genetic and/or epigenetic parameters of genes associated with pharmacogenomics by analyzing methylation patterns thereof. According to the present invention, the method is preferably used for the determination of genetic and/or epigenetic parameters of genes associated with pharmacogenomics.

The method according to the present invention is used, for example, for the diagnosis and/or therapy of solid tumours and cancer.

The nucleic acids according to the present invention of Seq. ID No.1 through Seq. ID No.174 and sequences complementary thereto and/or of a segment of the chemically pretreated DNA of genes associated with pharmacogenomics according to one of the sequences according to table 1 can be used for the determination of genetic and/or epigenetic parameters of genes associated with pharmacogenomics.

The present invention moreover relates to a method for manufacturing a diagnostic reagent and/or therapeutic agent for the diagnosis and/or therapy of diseases or of conditions associated with drug response by analyzing methylation patterns of genes associated with pharmacogenomics, the diagnostic agent and/or therapeutic agent being characterized in that at least one nucleic acid according to the present invention is used for manufacturing it, possibly together with suitable additives and auxiliary agents.

A further subject matter of the present invention relates to a diagnostic reagent and/or therapeutic agent for the diagnosis and/or therapy of diseases or of conditions associated with drug response by analyzing methylation patterns of genes associated with pharmacogenomics, the diagnostic agent and/or therapeutic agent containing at least one nucleic acid according to the present invention, possibly together with suitable additives and auxiliary agents.

The present invention moreover relates to the diagnosis and/or prognosis of events which are disadvantageous to patients or individuals in which important genetic and/or epigenetic parameters within genes associated with pharmacogenomics said parameters obtained by means of the present invention may be compared to another set of genetic and/or epigenetic parameters, the differences serving as the basis for a diagnosis and/or prognosis of events which are disadvantageous to patients or individuals.

In the context of the present invention, the term “pharmacogenonmics” encompasses the study of genetic variation underlying differential response to drugs, particularly genes involved in drug metabolism. The term further refers to the application of tools including, but not limited to, the functional genomics toolbox of differential gene expression (DGE), proteomics, yeast 2-hybrid (Y2H) analyses, tissue immuno- and histopathology, genotyping of SNPs and other polymorphisms, automated DNA sequencing, customised differential gene expression analysis, genostratification, and pharmacogenetic testing for variability in genes. Therefore, the application of modern genomic technologies, including SNPs, transcript profiling, and proteomics. SNPs may allow population “subgrouping^{t}” including the exclusion of patients who may have adverse responses to a drug or preselection of those who are most likely to benefit from a particular drug. They may also help in selection of clinical trial participants by providing better ways to determine whether a study group is truly heterogeneous or by allowing preselection of particular groups. Finally, pharmacogenomics involves the creation of individualized medicines based upon scientific and clinical data generated from a patient's genetic information. There are two applications of pharmacogenomics that may use similar techniques but are quite distinct: a) susceptibility gene identification and b) “right medicine for right patient” [Allen D. Roses “Pharmacogenetics and pharmacogenomics in the discovery and development of medicines ”Pharmacogenetique et Pharmacogenetique, Institut Pasteur, Paris [France], 12-13 Octobre 2000, Institut Pasteur]. In the present invention, pharmacogenomics is based on the differences in the methylation pattern between different copies of genes or genomes of individuals, e.g. patients.

In the context of the present invention the term “hybridization” is to be understood as a bond of an oligonucleotide to a completely complementary sequence along the lines of the Watson-Crick base pairings in the sample DNA, forming a duplex structure. To be understood by “stringent hybridization conditions” are those conditions in which a hybridization is carried out at 60° C. in 2.5×SSC buffer, followed by several washing steps at 37° C. in a low buffer concentration, and remains stable.

The term “functional variants” denotes all DNA sequences which are complementary to a DNA sequence, and which hybridize to the reference sequence under stringent conditions and have an activity similar to the corresponding polypeptide according to the present invention.

In the context of the present invention, “genetic parameters” are mutations and polymorphisms of genes associated with pharmacogenomics and sequences further required for their regulation. To be designated as mutations are, in particular, insertions, deletions, point mutations, inversions and polymorphisms and, particularly preferred, SNPs (single nucleotide polymorphisms).

In the context of the present invention, “epigenetic parameters” are, in particular, cytosine methylations and further chemical modifications of DNA bases of genes associated with pharmacogenomics and sequences further required for their regulation. Further epigenetic parameters include, for example, the acetylation of histones which, however, cannot be directly analyzed using the described method but which, in turn, correlates with the DNA methylation.

In the following, the present invention will be explained in greater detail on the basis of the sequences and examples with reference to the accompanying drawing without being limited thereto.

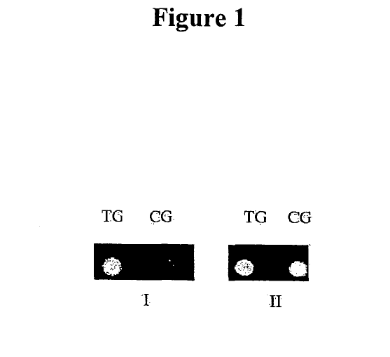

FIG. 1

FIG. 1 shows the hybridisation of fluorescent labelled amplificates to a surface bound olignonucleotide. Sample I being from a HT29 cell line cultured under standard conditions and sample II being from a HT29 cell line cultured under standard conditions with the addition of milrinone (1 μg/ml). Flourescence at a spot shows hybridisation of the amplificate to the olignonucleotide. Hybridisation to a CG olignonucleotide denotes methylation at the cytosine position being analysed, hybridisation to a TG olignonucleotide denotes no methylation at the cytosine position being analysed. It can be seen that Sample II had a higher degree of methylation than Sample I.

Seq. ID No. 1 trough Seq. ID No. 174

Sequences having odd sequence numbers (e.g., Seq. ID No. 1, 3, 5, . . . ) exhibit in each case sequences of the chemically pretreated genomic DNAs of different genes associated with pharmacogenomics. Sequences having even sequence numbers (e.g., Seq. ID No. 2, 4, 6, . . . ) exhibit in each case the sequences of the chemically pretreated genomic DNAs of genes associated with pharmacogenomics which are complementary to the preceding sequences (e.g., the complementary sequence to Seq. ID No. 1 is Seq. ID No.2, the complementary sequence to Seq. ID No.3 is Seq. ID No.4, etc.).

Seq. ID No. 175 trough Seq. ID No. 178

Seq. ID No. 1 trough Seq. ID No. 178 show sequences of oligonucleotides used in Example 1.

The following example relates to a fragment of a gene associated with pharmacogenomics, in this case, superoxide dismutase 1 in which a specific CG-position is analyzed for its methylation status.

### EXAMPLE 1

### Methylation Analysis of the Gene Superoxide Dismutase 1 Associated with Pharmacogenomics.

The following example relates to a fragment of the gene superoxide dismutase 1 in which a specific CG-position is to be analyzed for methylation.

Two samples of the cell line HT29 (human colon adenocarcinoma cell) were grown in culture. Sample 1 was cultured in a standard growth medium and Sample 2 was cultured an identical growth medium, with the addition of milrinone (1 μg/ml). The methylation status of the gene superoxide dismutase 1 was analysed in both samples.

In the first step, a genomic sequence is treated using bisulfite (hydrogen sulfite, disulfite) in such a manner that all cytosines which are not methylated at the 5-position of the base are modified in such a manner that a different base is substituted with regard to the base pairing behavior while the cytosines methylated at the 5-position remain unchanged.

If bisulfite solution is used for the reaction, then an addition takes place at the non-methylated cytosine bases. Moreover, a denaturating reagent or solvent as well as a radical interceptor must be present. A subsequent alkaline hydrolysis then gives rise to the conversion of non-methylated cytosine nucleobases to uracil. The chemically converted DNA is then used for the detection of methylated cytosines. In the second method step, the treated DNA sample is diluted with water or an aqueous solution. Preferably, the DNA is subsequently desulfonated at an alkaline pH value. In the third step of the method, the DNA sample is amplified in a polymerase chain reaction, preferably using a heat-resistant DNA polymerase. In the present case, cytosines of the gene superoxide dismutase 1 are analyzed. To this end, a defined fragment having a length of 451 bp is amplified with the specific primer oligonucleotides AGGGGAAGAAAAGGTAAGTT (Sequence ID 175) and CCCACTCTAACCCCAAACCA (Sequence ID No. 176). This amplificate serves as a sample which hybridizes to an oligonucleotide previously bonded to a solid phase, forming a duplex structure, for example TTTTGGGGCGTTTTAATT (Sequence ID No. 177), the cytosine to be detected being located at position 111 of the amplificate. The detection of the hybridization product is based on Cy3 and Cy5 fluorescently labelled primer oligonucleotides which have been used for the amplification. A hybridization reaction of the amplified DNA with the oligonucleotide takes place only if a methylated cytosine was present at this location in the bisulfite-treated DNA. Thus, the methylation status of the specific cytosine to be analyzed is inferred from the hybridization product.

In order to verify the methylation status of the position, a sample of the amplificate is further hybridized to another oligonucleotide previously bonded to a solid phase. Said olignonucleotide is identical to the oligonucleotide previously used to analyze the methylation status of the sample, with the exception of the position in question. At the position to be analysed said oligonucleotide comprises a thymine base as opposed to a cytosine base i.e TTTTGGGGTGTTTTAATT (Sequence ID No. 178). Therefore, the hybridisation reaction only takes place if an unmethylated cytosine was present at the position to be analysed.

### EXAMPLE 2

### Diagnosis of Diseases Associated with Pharmacogenomics

In order to relate the methylation patterns to one of the conditions associated with drug response, it is initially required to analyze the DNA methylation patterns of a group of affected and of a group of control patients. These analyses are carried out, for example, analogously to Example 1. The results obtained in this manner are stored in a database and the CpG dinucleotides which are methylated differently between the two groups are identified. This can be carried out by determining individual CpG methylation rates as can be done, for example, in a relatively imprecise manner, by sequencing or else, in a very precise manner, by a methylation-sensitive “primer extension reaction”. It is also possible for the entire methylation status to be analyzed simultaneously, and for the patterns to be compared, for example, by clustering analyses which can be carried out, for example, by a computer.

Subsequently, it is possible to allocate the examined patients to a specific therapy group and to treat these patients selectively with an individualized therapy.

TABLE 1
List of preferred genes associated with
pharmacogenomics according to the invention

| | |
|---|---|
| | Gen bank Entry No. |
| Gene | (http://www.ncbi.nlm.nih.gov) |
| ALDH6 | NM_000693 |
| CYP11A | NM_000781 |
| CYP11B1 | NM_000497 |
| CYP3A3 | NM_000776 & NM_017460 |
| DPYD | NM_000110 |
| EPHX2 | NM_001979 |
| OCLN | NM_002538 |
| TXNRD1 | NM_003330 |
| UGT8 | NM_003360 |
| MRP | NM_004996, NM_019900 |
| | NM_019901, NM_019902 |
| | NM_019862, NM_019898 & |
| | NM_019899 |

1. A nucleic acid comprising a sequence at least 18 bases in length of a segment of the chemically pretreated DNA of genes associated with pharmacogenomics according to one of the sequences taken from the group of Seq. ID No.1 to Seq. ID No.174 and sequences complementary thereto.

2. A nucleic acid comprising a sequence at least 18 base pairs in length of a segment of the chemically pretreated DNA of genes associated with pharmacogenomics according to one of the sequences of the genes ALDH6 (NM_{—}000693), CYP11A (NM_{—}000781), CYP11B1 (NM_{—}000497), CYP3A3 (NM_{—}000776 & NM_{—}017460), DPYD (NM_{—}000110), EPHX2 (NM_{—}001979), OCLN (NM_{—}002538), TXNRD1 (NM_{—}003330), UGTS (NM_{—}003360), MRP (NM_{—}004996, NM_{—}019900, NM_{—}019901, NM_{—}019902, NM_{—}019862, NM_{—}019898, NM019899) and sequences complementary thereto.

3. An oligomer, in particular an oligonucleotide or peptide nucleic acid (PNA)-oligomer, said oligomer comprising in each case at least one base sequence having a length of at least 9 nucleotides which hybridizes to or is identical to a chemically pretreated DNA of genes associated with pharmacogenomics according to one of the Seq ID Nos 1 to 174 according to claim 1 or to a chemically pretreated DNA of genes according to claim 2 and sequences complementary thereto.

4. The oligomer as recited in claim 3; wherein the base sequence includes at least one CpG dinucleotide.

5. The oligomer as recited in claim 3; characterized in that the cytosine of the CpG dinucleotide is located approximately in the middle third of the oligomer.

6. A set of oligomers, comprising at least two oligomers according to any of claims 3 to 5.

7. A set of oligomers as recited in claim 6, comprising oligomers for detecting the methylation state of all CpG dinucleotides within one of the sequences according to Seq. ID Nos. 1 through 174 according to claim 1 or a chemically pretreated DNA of genes according to claim 2, and sequences complementary thereto.

8. A set of at least two oligonucleotides as recited in claim 3, which can be used as primer oligonucleotides for the amplification of DNA sequences of one of Seq. ID 1 through Seq. ID 174 and sequences complementary thereto and/or sequences of a chemically pretreated DNA of genes according to claim 2, and sequences complementary thereto and segments thereof.

9. A set of oligonucleotides as recited in claim 8, characterized in that at least one oligonucleotide is bound to a solid phase.

10. Use of a set of oligomer probes comprising at least ten of the oligomers according to any of claims 6 through 9 for detecting the cytosine methylation state and/or single nucleotide polymorphisms (SNPs) in a chemically pretreated genomic DNA according to claim 1 or a chemically pretreated DNA of genes according to claim 2.

11. A method for manufacturing an arrangement of different oligomers (array) fixed to a carrier material for analyzing diseases associated with the methylation state of the CpG dinucleotides of one of the Seq. ID 1 through Seq. ID 174 and sequences complementary thereto and/or chemically pretreated DNA of genes according to claim 2, wherein at least one oligomer according to any of the claims 3 through 5 is coupled to a solid phase.

12. An arrangement of different oligomers (array) obtainable according to claim 11.

13. An array of different oligonucleotide- and/or PNA-oligomer sequences as recited in claim 12, characterized in that these are arranged on a plane solid phase in the form of a rectangular or hexagonal lattice.

14. The array as recited in any of the claims 12 or 13, characterized in that the solid phase surface is composed of silicon, glass, polystyrene, aluminium, steel, iron, copper, nickel, silver, or gold.

15. A DNA- and/or PNA-array for analyzing the methylation state of genes, comprising at least one nucleic acid according to one of the preceding claims.

16. A method for ascertaining genetic and/or epigenetic parameters for the diagnosis and/or therapy of existing diseases or the predisposition to specific diseases by analyzing cytosine methylations, characterized in that the following steps are carried out:
in a genomic DNA sample, cytosine bases which are unmethylated at the 5-position are converted, by chemical treatment, to uracil or another base which is dissimilar to cytosine in terms of hybridization behavior;
fragments of the chemically pretreated genomic DNA are amplified using sets of primer oligonucleotides according to claim 8 or 9 and a polymerase, the amplificates carrying a detectable label;
amplificates are hybridized to a set of oligonucleotides and/or PNA probes according to the claims 6 and 7, or else to an array according to one of the claims 12 through 15;
the hybridized amplificates are subsequently detected.

17. The method as recited in claim 16, characterized in that the chemical treatment is carried out by means of a solution of a bisulfite, hydrogen sulfite or disulfite.

18. The method as recited in one of the claims 16 or 17, characterized in that more than ten different fragments having a length of 100-2000 base pairs are amplified.

19. The method as recited in one of the claims 16 through 18, characterized in that the amplification of several DNA segments is carried out in one reaction vessel.

20. The method as recited in one of the claims 16 through 19, characterized in that the polymerase is a heat-resistant DNA polymerase.

21. The method as recited in claim 20, characterized in that the amplification is carried out by means of the polymerase chain reaction (PCR).

22. The method as recited in one of the claims 16 through 21, characterized in that the labels of the amplificates are fluorescence labels.

23. The method as recited in one of the claims 16 through 21, characterized in that the labels of the amplificates are radionuclides.

24. The method as recited in one of the claims 16 through 21, characterized in that the labels of the amplificates are detachable molecule fragments having a typical mass which are detected in a mass spectrometer.

25. The method as recited in one of the claims 16 through 21, characterized in that the amplificates or fragments of the amplificates are detected in the mass spectrometer.

26. The method as recited in one of the claims 24 and/or 25, characterized in that the produced fragments have a single positive or negative net charge for better detectability in the mass spectrometer.

27. The method as recited in one of the claims 24 through 26, characterized in that detection is carried out and visualized by means of matrix assisted laser desorption/ionization mass spectrometry (MALDI) or using electron spray mass spectrometry (ESI).

28. The method as recited in one of the claims 16 through 27, characterized in that the genomic DNA is obtained from cells or cellular components which contain DNA, sources of DNA comprising, for example, cell lines, biopsies, blood, lymphatic fluid, sputum, stool, urine, cerebral-spinal fluid, tissue embedded in paraffin such as tissue from eyes, intestine, kidney, brain, heart, prostate, lung, breast or liver, histologic object slides, and all possible combinations thereof.

29. A kit comprising a bisulfite (=disulfite, hydrogen sulfite) reagent as well as oligonucleotides and/or PNA-oligomers according to one of the claims 3 through 5.

30. The use of a nucleic acid according to claims 1 or 2, of an oligonucleotide or PNA-oligomer according to one of the claims 3 through 5, of a kit according to claim 29, of an array according to one of the claims 12 through 15, of a set of oligonucleotides according to one of claims 6 through 9 for the diagnosis of diseases.

31. The use of a nucleic acid according to claims 1 or 2, of an oligonucleotide or PNA-oligomer according to one of claims 3 through 5, of a kit according to claim 29, of an array according to one of the claims 12 through 15, of a set of oligonucleotides according to one of claims 6 through 9 for the therapy of diseases.
